(19) United States
(12) Patent Application Publication
(10) Pub. No.: US 2025/0231160 A1
(43) Pub. Date: Jul. 17, 2025

(54) HYDROGEN SENSING WITH THERMAL COMPENSATION

(71) Applicant: Baker Hughes Oilfield Operations LLC, Houston, TX (US)

(72) Inventors: Darin Duphorne, Houston, TX (US); Paul Wysocki, Blacksburg, VA (US); Zhao Li, Blacksburg, VA (US); Dan Provenzano, Blacksburg, VA (US)

(21) Appl. No.: 18/409,993

(22) Filed: Jan. 11, 2024

(51) Int. Cl. G01N 33/00; E21B 47/07; G01N 21/17; G01N 21/55

(52) U.S. Cl. CPC G01N 33/0032; E21B 47/07; G01N 21/55; G01N 2021/1789; G01N 2201/0886; G01N 2201/12746

(57) ABSTRACT

A system and method for determining a partial pressure of hydrogen in a volume. A response is measured of a section of an optical fiber disposed in the volume to a parameter of the volume. A partial pressure of hydrogen in the volume is determined from the response of the optical fiber to the parameter. The presence of hydrogen in the volume is determined from the partial pressure of hydrogen.

### BACKGROUND

Oil and gas recovery systems, fluid sequestration systems, hydrogen storage and hydrogen transport systems can operate in environment in which hydrogen can be present. The effectiveness of the system can be reduced in the presence the hydrogen. For example, hydrogen can cause metal to degrade. Optical fibers that are used in these systems for optical communication of sensing can darken in the presence of hydrogen, causing a strength of an optical signal propagating through the optical fiber to be reduced. Accordingly, there is a need to be able to detect the presence, quantity, and history of hydrogen in the system in order to take preventative measures.

### SUMMARY

Disclosed herein is a method of determining a partial pressure of hydrogen in a volume. A calibration signal characteristic is obtained along an optical fiber in an absence of hydrogen, wherein the calibration signal characteristic is associated with a presence of hydrogen. The optical fiber is disposed in the volume. A temperature profile is measured along a section of the optical fiber. A measured signal characteristic is obtained along the section of the optical fiber. The partial pressure of hydrogen in the volume is determined from a comparison of the measured signal characteristic to the calibrated signal characteristic.

Also disclosed herein is a system of determining a partial pressure of hydrogen in a volume. The system includes an optical fiber, a light source for generating a light at a selected wavelength for propagating through the optical fiber, a light sensor for measuring the light after propagating through the optical fiber, and a processor. The processor is configured to obtain a calibration signal characteristic along the optical fiber in an absence of hydrogen, wherein the calibration signal characteristic is associated with a presence of hydrogen, measure a temperature profile along a section of the optical fiber with the optical fiber disposed in the volume, obtain a measured signal characteristic along the section of the optical fiber with the optical fiber in the volume, and determine the partial pressure of hydrogen in the volume from a comparison of the measured signal characteristic to the calibrated signal characteristic.

Also disclosed herein is a method of determining a presence of hydrogen in a volume. A response is measured of a section of an optical fiber disposed in the volume to a parameter of the volume. A partial pressure of hydrogen in the volume is determined from the response of the optical fiber to the parameter. The presence of hydrogen in the volume is determined from the partial pressure of hydrogen.

Also disclosed herein is a system for determining a partial pressure of hydrogen in a volume. The system includes an optical fiber disposed in the volume, a light source for generating a light at a selected wavelength for propagating through the optical fiber, a light sensor for measuring the light after propagating through the optical fiber, and a processor. The processor is configured to measure a response of a section of the optical fiber to a parameter of the volume, determine the partial pressure of hydrogen in the volume from the response of the optical fiber to the parameter, and determine a presence of hydrogen in the volume from the partial pressure of hydrogen.

### BRIEF DESCRIPTION OF THE DRAWINGS

The following descriptions should not be considered limiting in any way. With reference to the accompanying drawings, like elements are numbered alike:

FIG. 1 discloses a borehole system in an illustrative embodiment;

FIG. 2 shows a graph of signal absorption or signal loss at various wavelengths of light propagating in the optical fiber;

FIG. 3 shows an expanded view of the graph of FIG. 2 in a wavelength region between 1460 nanometers and 1580 nanometers;

FIG. 4 is a graph depicting a relation between proportionality constants and temperature at various wavelengths;

FIG. 5 shows a flowchart of a method for determining a hydrogen concentration at a depth in a borehole using a signal loss of light propagating through an optical fiber in the borehole;

FIG. 6 shows a borehole system for obtaining measurements at a plurality of depths, in an illustrative embodiment;

FIG. 7 shows a borehole system for obtaining measurements over a range of locations, in an illustrative embodiment;

FIG. 8 shows a borehole system in an alternative embodiment;

FIG. 9 shows a borehole system in an alternative embodiment; and

FIG. 10 shows a hydrogen sensing system in an embodiment.

### DETAILED DESCRIPTION

A detailed description of one or more embodiments of the disclosed apparatus and method are presented herein by way of exemplification and not limitation with reference to the Figures.

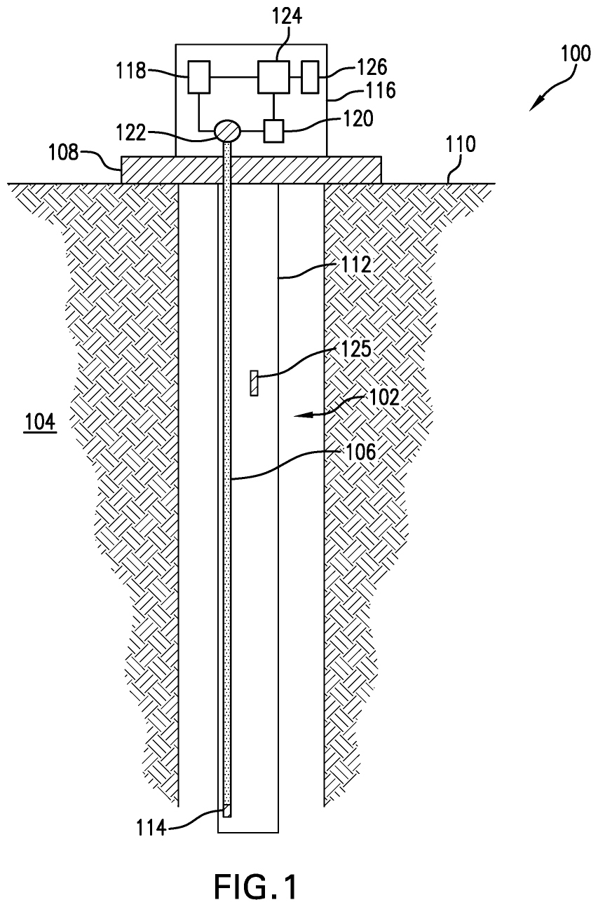

Referring to FIG. 1, a borehole system 100 is shown in an illustrative embodiment. The borehole system 100 includes a borehole 102 formed in a subsurface formation 104. An optical fiber 106 extends into the borehole 102 from a platform 108 at a surface location 110. The optical fiber 106 can be disposed in the borehole 102 separately or as a part of a work string 112. The work string 112 can be any type of string used in a borehole 102 such as, for example, a drill string, a production string, a stimulation string, a sequestration string, etc. A reflector 114 is located at a bottom end of the optical fiber 106.

In various embodiments, the optical fiber 106 is sensitive to the presence of hydrogen. In particular, the optical fiber 106 can absorb hydrogen, resulting in a signal loss or loss of intensity for light propagating through the optical fiber 106. The signal loss is temporary (not permanent) and dissipates when the hydrogen is no longer present (i.e., the absorption loss returns to a nominal absorption of the optical fiber). In various embodiments, the optical fiber 106 is a CoreBright™ optical fiber of Baker Hughes Incorporated. However, any optical fiber that experiences temporary signal loss in the presence of hydrogen can be used. An ideal optical fiber is one that experiences only a temporary loss when exposed to hydrogen (i.e., the loss occurs in the presence of hydrogen but goes away when the hydrogen is removed or is no longer present). An optical fiber that exhibits permanent loss when exposed to hydrogen is less advantageous because the loss does not go away when the hydrogen is removed, which reduces the ability to compute the amount of hydrogen present over time. Permanent darkening might also inhibit the ability to receive signals through or reflected from the fiber. If an ideal fiber is selected (for example, CoreBright™), the amount of light absorption or signal loss at a given location in the optical fiber 106 is directly proportional to an amount of hydrogen at the given location. Thus, the optical fiber 106 can be used as a hydrogen sensor.

A controller 116 is located at the platform 108. The controller 116 includes a laser or light source 118, a light detector or light sensor 120, a circulator 122, a processor 124, and a memory storage device 126. The light source 118 generates the light at a selected frequency. In various embodiments, the light source 118 can be tunable to transmit light at one or more frequencies or over a range of frequencies. Alternatively, the light source 118 can include multiple light sources that can be used to transmit light at different frequencies. In various embodiments, the light source 118 can generate light at 1550 nanometers (nm) and/or at 1470 nm, for example. The processor 124 can access instructions stored in the memory storage device 126 and execute the instructions to perform various methods disclosed herein. The processor 124 can control the light source 118 to perform various optical testing methods, such as optical time domain reflectometry (OTDR), optical frequency domain reflectometry (OFDR), distributed temperature sensing (DTS), etc. OTDR can be used to characterize the optical fiber and identify a signal characteristic along the optical fiber. In various embodiments disclosed herein, the signal characteristic is a signal loss or a signal loss profile along the optical fiber. However, the signal characteristic can be a pattern of reflection or pattern of refraction in alternative embodiments. DTS can be used to measure a temperature profile along the optical fiber.

The processor 124 operates the light source 118 to control various parameters of the light (e.g., wavelength, pulse width). The light source 118 generates light at a selected wavelength, and the circulator 122 circulates the light into the optical fiber 106. The light propagates downhole and is reflected either along the optical fiber 106 or at the reflector 114 to propagate back to the controller 116. The circulator 122 circulates the returning light to the light sensor 120. The light sensor 120 detects the returning light and reads an optical parameter (e.g., a temperature, a signal loss) of the returning light. As the light propagates through the optical fiber, signal loss can occur due to the presence of hydrogen at one or more locations or depths in the borehole 102. In an embodiment, the processor 124 determines a temperature in the borehole 102 and a signal loss of the light propagating in the optical fiber 106 due to the presence of hydrogen. The processor 124 determines a partial pressure of hydrogen in the borehole based on the signal loss and the temperature in the borehole 102.

To determine the hydrogen partial pressure (partial pressure of hydrogen), a calibrated signal loss is determined for the optical fiber 106 before the optical fiber 106 is disposed in the borehole. This calibration can be performed in a laboratory setting, for example, or any setting with an absence of hydrogen. Once the optical fiber 106 is disposed in the borehole, a temperature profile and a signal loss profile are measured. The signal loss is compared to the calibrated signal loss and normalize with a suitable proportionality constant based on the temperature, as disclosed herein.

A pressure gauge 125 can be disposed at a given location in the borehole to measure a total pressure at the given location in the borehole. The processor 124 can determine a hydrogen concentration at the location from the partial pressure of hydrogen and the measured borehole pressure at the location, specifically from a ratio of the hydrogen partial pressure to the total pressure.

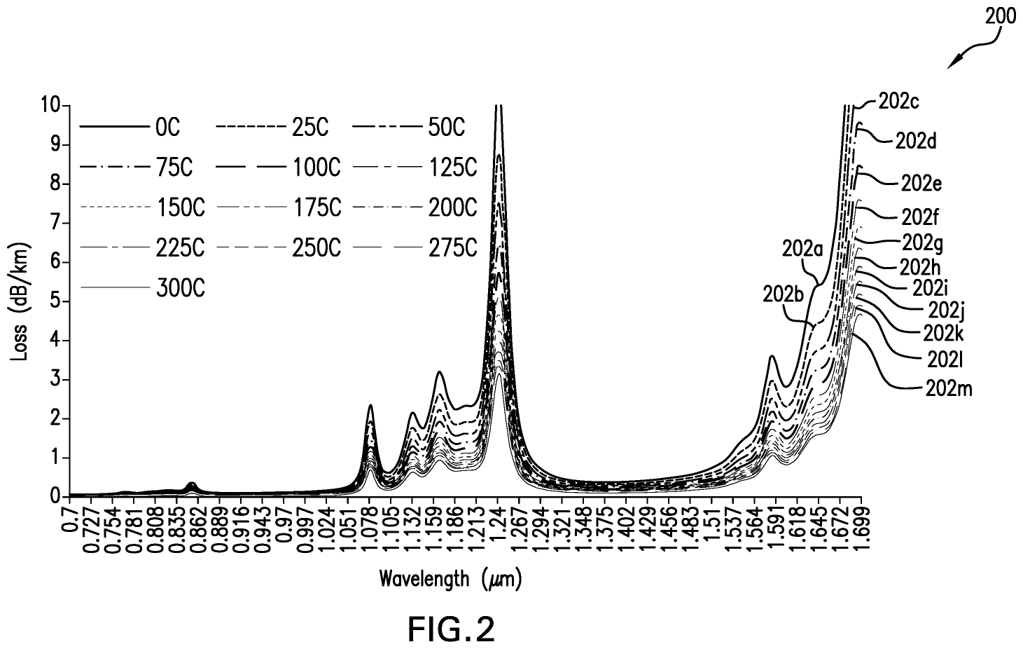

FIG. 2 shows a graph 200 of signal absorption or signal loss at various wavelengths of light propagating in the optical fiber 106. Wavelength is shown along the abscissa in micrometers (μm) and signal loss is shown along the ordinate axis in decibels per kilometer (dB/km). Signal absorption is shown in a wavelength region between about 700 nm to about 1700 nm. The absorption curves 202a-202m are obtained for the optical fiber in an environment without hydrogen and illustrate the effects of temperature on signal loss at various wavelengths (when no hydrogen is present). The signal loss is measured at various temperatures within a typical range of temperatures encountered in borehole applications. For illustrative purposes, the absorption curves 202a-202m range between zero degrees Celsius and 300 degrees Celsius. (i.e., 0° C., 25° C., 50° C., 75° C., 100° C., 125° C., 150° C., 175° C., 200° C., 225° C., 250° C., 275° C., and 300° C., respectively). As shown in FIG. 2, the signal absorption at each wavelength is dependent on temperature and increases with temperature. For each curve, absorption peaks occur at about 1.24 μm and 1.59 μm.

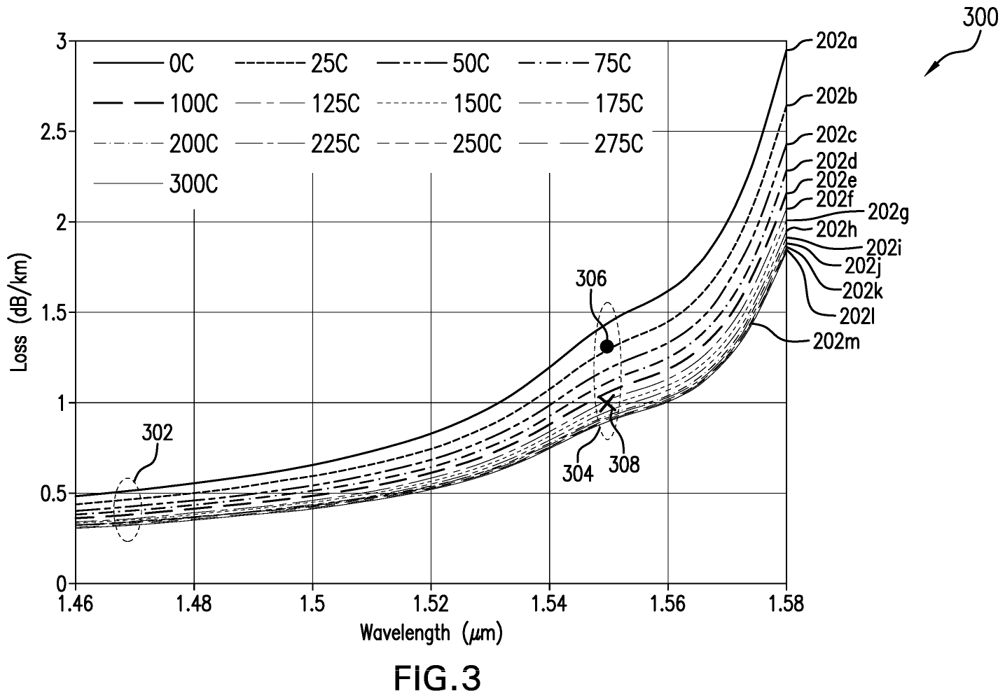

FIG. 3 shows an expanded view 300 of the graph 200 of FIG. 2 in a wavelength region between 1460 nm and 1580 nm. In the expanded view 300, the signal loss is shown to be substantially different at 1470 nm (indicated by first ellipse 302) than at 1550 nm (indicated by second ellipse 304). Thus, signal loss measurements at 1470 nm and 1550 nm (when the optical fiber 106 is in a borehole) can be used to determine when a change in signal loss is due to the presence of hydrogen or due to a change in temperature. For example, a change in temperature affects the signal loss at both wavelengths, while an ingress of hydrogen into the borehole affects only the signal loss at 1550 nm (or affects the signal loss at 1550 nm more than the signal loss at 1470 nm).

A calibrated signal loss 306 (laboratory measurement) and a measured signal loss 308 (when the optical fiber 106 is in the borehole 102) are shown in FIG. 3. For illustrative purposes, the calibrated signal loss is about 1.25 dB/km and the measured signal loss is about 1.0 dB/km. The calibrated signal loss 306 is obtained for light having a wavelength of 1550 nm traveling in the optical fiber 106 at a temperature of 25 C and a pressure of 1 atm. The light has a wavelength of 1550 nm and the downhole (borehole) temperature is 25 C. The partial pressure of hydrogen in the borehole is unknown but can be determined from a comparison of the calibrated signal loss 306 and the measured signal loss 308.

In particular, the downhole pressure can be determined from a ratio of a difference between the calibrated signal loss and the measured signal loss to a proportionally constant. The proportionality constant relates signal loss in an optical fiber to a hydrogen pressure. The proportionality constant K(λ,T) has units of dB/km per Atm [dB/km/Atm] or of dB/km per psi of Hydrogen [dB/km/psi]. The proportionality constant can be determined before the optical fiber 106 is deployed in the borehole and can then be stored in the memory storage device 126 for subsequent access by the processor 124. The proportionality constant might also be approximated using a theoretical understanding of the physics involved and known glass properties.

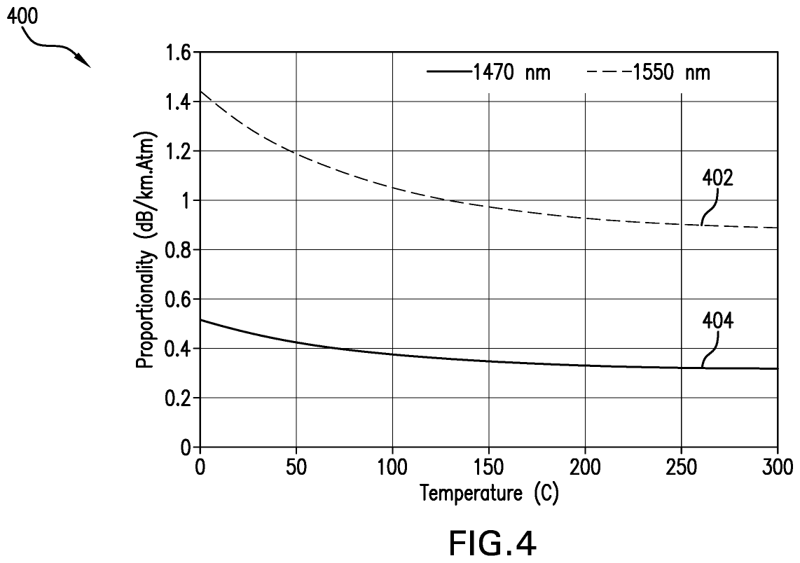

FIG. 4 is a graph 400 depicting a relation between proportionality constants and temperature at various wavelengths. A first curve 402 indicates the proportionality constant for light having a wavelength of 1550 nm. A second curve 404 indicates the proportionality constant for light having a wavelength of 1470 nm. Each of the first curve 402 and the second curve 404 changes with temperature. A value of the proportionality constant can be selected based on a temperature measurement in the borehole.

To determine the partial pressure of hydrogen along an extended length or continuous length of the borehole, a calibrated signal loss α_{base}(λ,z) is obtained using OTDR measurements at a desired wavelength (e.g., 1550 nm) in an environment having no hydrogen present. The proportionality constant K(λ,T(z)) is determined, for example, from the relation shown in FIG. 3 using the measured temperature and the light wavelength. The optical fiber 106 is then deployed in in the borehole and a signal loss profile α(λ,T(z),P(z),z) is measured (using OTDR) and temperature T(z) is measured (using DTS) along a length z of the optical fiber 106 for light at the desired wavelength. The measured signal loss is dependent on the temperature at depth z, pressure at depth z, and the wavelength of light. The partial pressure of hydrogen can then be determined along length z using Eq. (1):

$$P ( z ) = \frac{[ ∝ ( λ , T ( z ) , P ( z ) , z ) - {α}_{base} ( λ , z ) ]}{K ( λ , T ( z ) )} \qquad ( Eq . 1 )$$

where P(z) is the hydrogen pressure at a selected depth z.

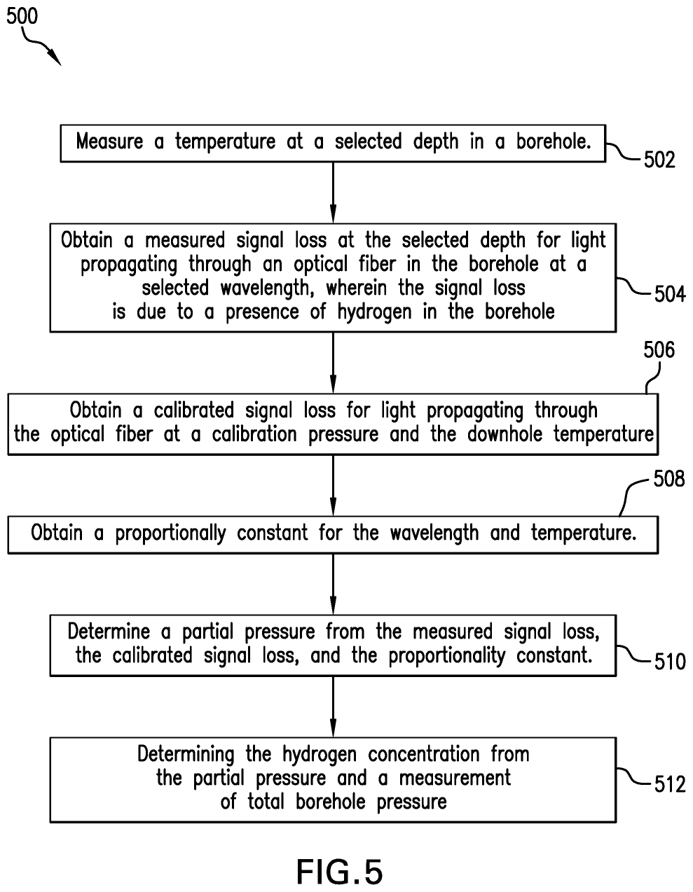

FIG. 5 shows a flowchart 500 of a method for determining a hydrogen partial pressure in a borehole 102. In box 502, a calibration signal loss is measured for an optical fiber that is disposed in an environment having no hydrogen present or an insignificant amount of hydrogen present. The calibration signal loss can be obtained for light propagating at a single wavelength, at two wavelengths of light (e.g., 1550, or 1550 and 1470) or at more wavelengths.

In box 504, a proportionality constant is determined for the optical fiber. The proportionality constant relates signal loss at a given pressure to temperature. In box 506, the optical fiber is disposed in a borehole. In box 508, a distributed temperature profile is measured along a length of the optical fiber (and thus along a length of the borehole). In box 510, a signal loss is measured along the length of the optical fiber. The signal loss measurement can be obtained simultaneously with the temperature measurement or soon thereafter. Thus, the temperature profile obtained in box 508 is concurrent with the signal loss profile of box 510. In box 512, a partial pressure of hydrogen in the borehole is determined from the calibrated signal loss (box 502), the measured signal loss (box 510), the temperature profile (box 508), and the proportionality constant (box 504), using Eq. (1). In box 514, a hydrogen concentration in the borehole 102 is determined from the partial pressure of hydrogen and the total pressure obtained via the pressure gauge 125.

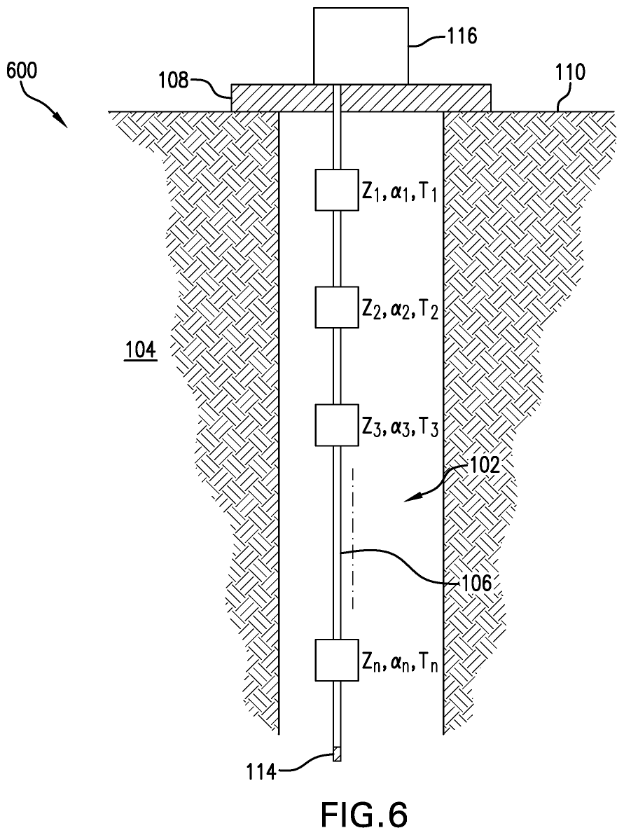

With the optical fiber disposed along a length of the borehole, the temperature and signal loss measurements are distributed measurements made along a given length of the borehole. Thus, the hydrogen partial pressure is determined along this length. The optical fiber can also be used to make localized measurements by forming loops in the optical fiber, as discussed herein with respect to FIG. 6.

FIG. 6 shows a borehole system 600 for obtaining measurements at a plurality of depths, in an illustrative embodiment. Depths (z₁, z₂, . . . , zₙ) are shown at a plurality of discrete depths within the borehole. As an illustrative example, the optical fiber has 10 loops that are 100 m in length with the loops separated by 100 m. Each depth includes a loop in which a section of the fiber is rolled up to a coil. When a section is coiled into a loop, measurements from the section pertains only to the depth at which the loop is disposed (e.g., depth z₁). The loop can be any selected length. Looping the optical fiber 106 increases the reliability of measurements of the signal loss at the selected depth. It should also be noted that, in such a case, one might be able to use a single discrete temperature measurement at each localized depth while maintaining the distributed nature of the hydrogen measurement. Such a temperature measurement can be provided by a discrete optical temperature measuring device or by another technology. Even without the loops, such discrete measurements can be used to provide the temperature data necessary to properly calibrate the hydrogen measurement.

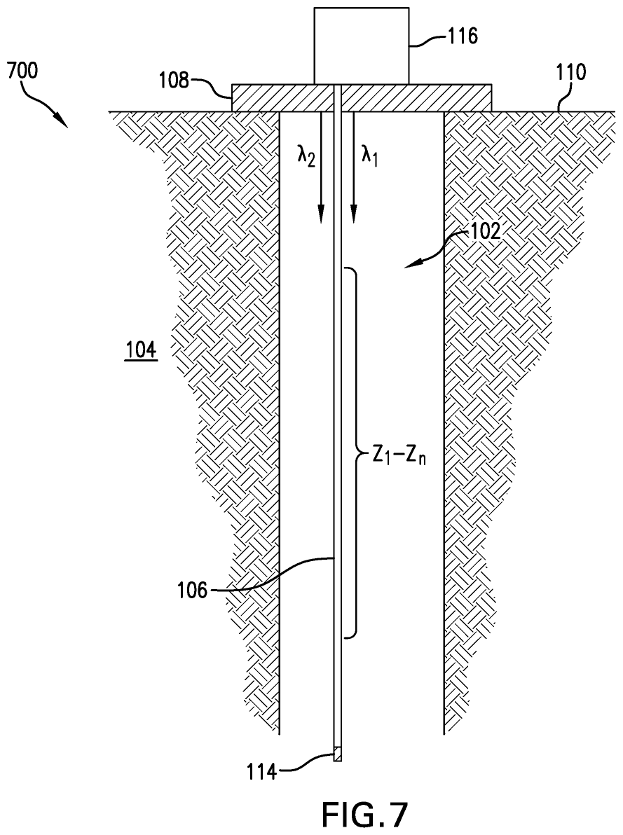

FIG. 7 shows a borehole system 700 for obtaining measurements over a range of locations using a plurality of light wavelengths, in an illustrative embodiment. In an embodiment, a first light having a first wavelength Au and a second light having a second wavelength λ₂ can be propagated through the optical fiber 106 to obtain two sets of temperature and signal loss measurements. A first hydrogen partial pressure can be determined from the first set of measurements and a second hydrogen partial pressure can be determined form the second set of measurements. The second hydrogen partial pressure can be used to verify or cross-check the first hydrogen partial pressure.

In one embodiment, DTS is performed using multiple wavelengths and computes temperature using signals at the multiple wavelengths. These wavelengths are also used to measure the loss of the fiber due to the presence of hydrogen. Thus, the wavelengths are suitable for sensing the presence of hydrogen. For example, it is possible to make a DTS measurement and measuring scattering at both 1470 nm and 1550 nm, and a Raman signal generated at 1470 nm by light at 1550 nm. Thus, the wavelengths are used to provide not only temperature along a fiber length but also a change in signal loss at 1550 nm due to hydrogen ingress. The signal at 1470 nm sees less loss due to hydrogen and so can be used to differentiate any losses that are not due to hydrogen ingress.

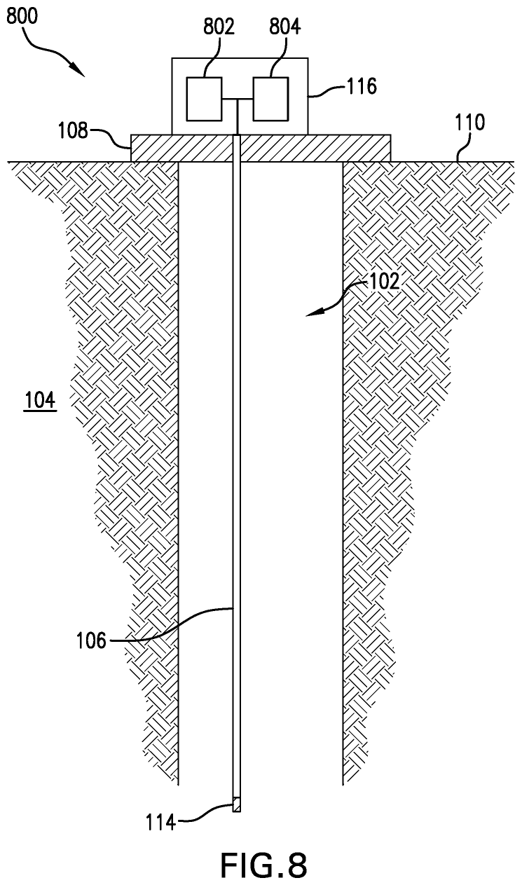

FIG. 8 shows a borehole system in 800 an alternative embodiment. The controller 116 includes a first optical interrogation unit 802 for performing OTDR and a second optical interrogation unit 804 for performing DTS. In an embodiment, the first optical interrogation unit 802 performs single mode OTDR and the second optical interrogation unit 804 performs single mode DTS. Alternatively, the first optical interrogation unit 802 performs single mode OTDR and the second optical interrogation unit 804 performs multimode DTS.

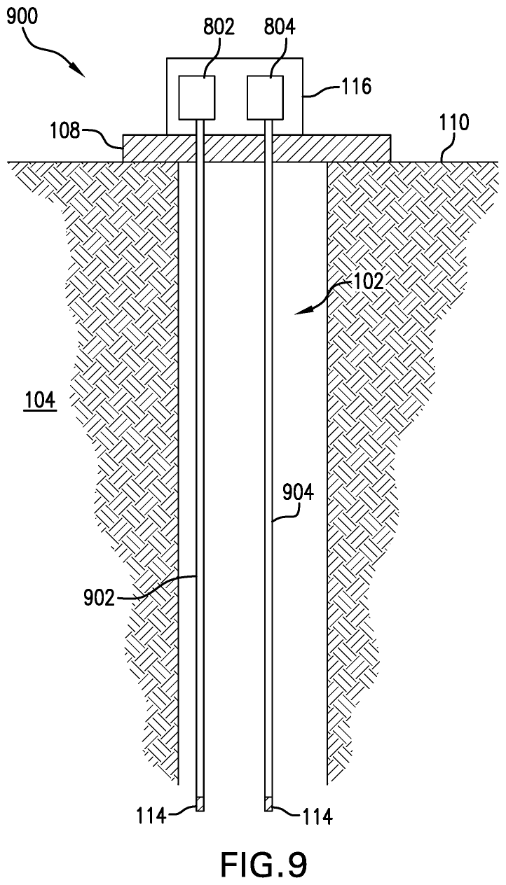

FIG. 9 shows a borehole system 900 in an alternative embodiment. The controller 116 includes a first optical fiber 902 and a second optical fiber 904, both extending into the borehole. The first optical interrogation unit 802 performs OTDR over the first optical fiber 902 and the second optical interrogation unit 804 performs DTS over the second optical fiber 904.

Although the system and methods disclosed herein are discussed with respect to a borehole, it is understood that the system and methods can be applied to any volume or any enclosed volume. Exemplary volumes include the borehole, a pipeline at a surface location, a hydrogen storage facility, etc. The optical fiber can detect any parameter of the volume (including temperature, pressure) and a partial pressure of hydrogen can be determined based on a response of the optical fiber to the parameter.

The presence of hydrogen, or a concentration of hydrogen in the volume can indicate a failure of a component of the volume, such as a degradation of a metal of a pipeline or hydrogen storage facility, or the delivery of hydrogen through the pipeline.

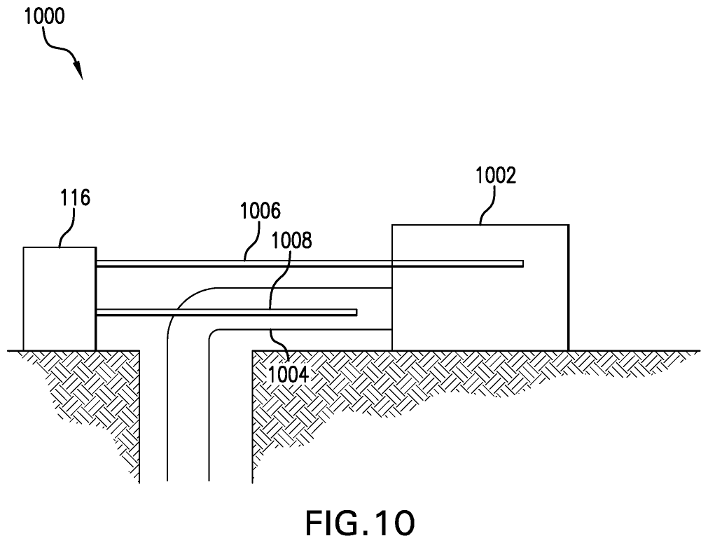

FIG. 10 shows a hydrogen sensing system 1000 in an embodiment. The hydrogen sensing system 1000 determine a presence of hydrogen in a hydrogen storage facility 1002 and/or a pipeline 1004. The controller 116 has a first optical fiber 1006 extending into the hydrogen storage facility 1002 and/or a second optical fiber 1008 extending into the pipeline 1004. The controller 116 obtains signal loss from the first optical fiber 1006 and the second optical fiber 1008 as disclosed herein. The hydrogen storage facility 1002 and/or the pipeline 1004 and be monitored at a pressure as high as about 300 atmospheres. In addition, DTS and DAS can be performed to determine the presence of leaks. A warning signal can be generated to indicate the presence of hydrogen in any of the systems disclosed herein, including the hydrogen storage facility 1002 and the pipeline 1004. A signal can also be displayed to indicate the partial presence of hydrogen in the volume.

A failure in any of the systems disclosed herein can be caused by the cumulative effects of hydrogen over time at a given temperature. The hydrogen sensing systems described herein can be used to track the total hydrogen exposure at each location by integrating the signal loss measurements over time. Such a hydrogen sensing system that provides both temperature measurements and hydrogen levels as a function of position and time can be used in conjunction with measurements of other parameters, such as distributed strain, that might be used to predict hydrogen-related failures or hydrogen-related performance degradation in more complex systems.

In general, the optical fiber can be disclosed in a volume and a response of the optical fiber to a parameter of the volume can be measured. A partial pressure of hydrogen in the volume is determined based on the response to the parameter. The presence of hydrogen in the volume (i.e., a concentration of hydrogen in the volume) is determined from the partial pressure of hydrogen.

In an embodiment, a temporal response of the optical fiber to the parameter can be measured over time. A temporal pattern in the response of the optical fiber to the parameter can be determined. A temporal pattern of the hydrogen concentration in the volume can be determined from the temporal pattern of the response.

Set forth below are some embodiments of the foregoing disclosure:

Embodiment 1. A method of determining a partial pressure of hydrogen in a volume. A calibration signal characteristic is obtained along an optical fiber in an absence of hydrogen, wherein the calibration signal characteristic is associated with a presence of hydrogen. The optical fiber is disposed in the volume. A temperature profile is measured along a section of the optical fiber. A measured signal characteristic is obtained along the section of the optical fiber. The partial pressure of hydrogen in the volume is determined from a comparison of the measured signal characteristic to the calibrated signal characteristic.

Embodiment 2. The method of any prior embodiment, further comprising determining the partial pressure of hydrogen from a ratio of a difference of the calibrated signal characteristic and the measured signal characteristic to a proportionality constant.

Embodiment 3. The method of any prior embodiment, wherein the proportionality constant relates signal characteristic to temperature.

Embodiment 4. The method of any prior embodiment, wherein the volume comprises a borehole, further comprising measuring the temperature profile and measuring the measured signal characteristic at one of: (i) along a continuous length of the borehole; (ii) a single depth in the borehole; and (iii) a plurality of discrete depths in the borehole.

Embodiment 5. The method of any prior embodiment, wherein the volume comprises a borehole, further comprising forming a loop in the optical fiber at a selected depth of the borehole and determining the partial pressure of hydrogen at the selected depth using the measured signal characteristic and a temperature at the selected depth obtained from the loop.

Embodiment 6. The method of any prior embodiment, further comprising propagating a first light through the optical fiber at a first wavelength and a second light through the optical fiber at a second wavelength and determining a first hydrogen partial pressure based on the first light and a second hydrogen partial pressure based on the second light.

Embodiment 7. The method of any prior embodiment, further comprising measuring the measured signal characteristic using Optical Time Domain Reflectometry (OTDR) and measuring the temperature profile using Distributed Temperature Sensing (DTS).

Embodiment 8. The method of any prior embodiment, wherein the volume comprises a borehole, further comprising one of: (i) performing OTDR using a first optical interrogation unit and performing DTS using a second optical interrogation unit; (ii) performing OTDR using a single mode unit and performing DTS using a multimode unit; (iii) performing DTS along a first optical fiber in the borehole and performing OTDR along a second optical fiber in the borehole.

Embodiment 9. The method of any prior embodiment, wherein the signal characteristic is a signal loss.

Embodiment 10. A system of determining a partial pressure of hydrogen in a volume. The system includes an optical fiber, a light source for generating a light at a selected wavelength for propagating through the optical fiber, a light sensor for measuring the light after propagating through the optical fiber, and a processor. The processor is configured to obtain a calibration signal characteristic along the optical fiber in an absence of hydrogen, wherein the calibration signal characteristic is associated with a presence of hydrogen, measure a temperature profile along a section of the optical fiber with the optical fiber disposed in the volume, obtain a measured signal characteristic along the section of the optical fiber with the optical fiber in the volume, and determine the partial pressure of hydrogen in the volume from a comparison of the measured signal characteristic to the calibrated signal characteristic.

Embodiment 11. The system of any prior embodiment, wherein the processor is further configured to determine the partial pressure of hydrogen from a ratio of a difference of the calibrated signal characteristic and the measured signal characteristic to a proportionality constant.

Embodiment 12. The system of any prior embodiment, wherein the volume comprises a borehole and the processor is further configured to measure the temperature profile and obtain the measured signal characteristic at one of: (i) along a continuous length of the borehole; (ii) a single depth in the borehole; and (iii) a plurality of discrete depths in the borehole.

Embodiment 13. The system of any prior embodiment, wherein the volume comprises a borehole and the optical fiber includes a loop at a selected depth of the borehole and wherein the processor is further configured to determine the partial pressure of hydrogen at the selected depth using the measured signal characteristic and the temperature at the selected depth obtained from the loop.

Embodiment 14. The system of any prior embodiment, wherein the light source is further configured to propagate a first light through the optical fiber at a first wavelength and a second light through the optical fiber at a second wavelength and the processor is further configured to determine a first partial pressure of hydrogen based on the first light and a second partial pressure of hydrogen based on the second light.

Embodiment 15. The system of any prior embodiment, wherein the processor is further configured to obtain the measured signal characteristic using Optical Time Domain Reflectometry (OTDR) and measure the temperature using Distributed Temperature Sensing (DTS).

Embodiment 16. The system of any prior embodiment, wherein the volume comprises a borehole and the processor is further configured to perform one of: (i) OTDR using a first optical interrogation unit and DTS using a second optical interrogation unit; (ii) OTDR using a single mode unit and DTS using a multimode unit; (iii) DTS along a first optical fiber in the borehole and OTDR along a second optical fiber in the borehole.

Embodiment 17. A method of determining a presence of hydrogen in a volume. A response is measured of a section of an optical fiber disposed in the volume to a parameter of the volume. A partial pressure of hydrogen in the volume is determined from the response of the optical fiber to the parameter. The presence of hydrogen in the volume is determined from the partial pressure of hydrogen.

Embodiment 18. The method of any prior embodiment, wherein determining the presence of hydrogen further comprises determining a concentration of hydrogen in the volume from the partial pressure of hydrogen.

Embodiment 19. The method of any prior embodiment, further comprising measuring a temporal response of the optical fiber to the parameter and determining a temporal pattern of the hydrogen concentration in the volume from the temporal response.

Embodiment 20. The method of any prior embodiment, wherein the parameter is one of: (i) temperature of the volume; and (ii) pressure of the volume.

Embodiment 21. A system for determining a partial pressure of hydrogen in a volume. The system includes an optical fiber disposed in the volume, a light source for generating a light at a selected wavelength for propagating through the optical fiber, a light sensor for measuring the light after propagating through the optical fiber, and a processor. The processor is configured to measure a response of a section of the optical fiber to a parameter of the volume, determine the partial pressure of hydrogen in the volume from the response of the optical fiber to the parameter, and determine a presence of hydrogen in the volume from the partial pressure of hydrogen.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The terms “about”, “substantially” and “generally” are intended to include the degree of error associated with measurement of the particular quantity based upon the equipment available at the time of filing the application. For example, “about” and/or “substantially” and/or “generally” can include a range of ±8% a given value.

The teachings of the present disclosure may be used in a variety of well operations. These operations may involve using one or more treatment agents to treat a formation, the fluids resident in a formation, a borehole, and/or equipment in the borehole, such as production tubing. The treatment agents may be in the form of liquids, gases, solids, semi-solids, and mixtures thereof. Illustrative treatment agents include, but are not limited to, fracturing fluids, acids, steam, water, brine, anti-corrosion agents, cement, permeability modifiers, drilling muds, emulsifiers, demulsifiers, tracers, flow improvers etc. Illustrative well operations include, but are not limited to, hydraulic fracturing, stimulation, tracer injection, cleaning, acidizing, steam injection, water flooding, cementing, etc.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A method of determining a partial pressure of hydrogen in a volume, comprising:
obtaining a calibration signal characteristic along an optical fiber in an absence of hydrogen, wherein the calibration signal characteristic is associated with a presence of hydrogen;
disposing the optical fiber in the volume;
measuring a temperature profile along a section of the optical fiber;
obtaining a measured signal characteristic along the section of the optical fiber; and
determining the partial pressure of hydrogen in the volume from a comparison of the measured signal characteristic to the calibrated signal characteristic.

2. The method of claim 1, further comprising determining the partial pressure of hydrogen from a ratio of a difference of the calibrated signal characteristic and the measured signal characteristic to a proportionality constant.

3. The method of claim 2, wherein the proportionality constant relates signal characteristic to temperature.

4. The method of claim 1, wherein the volume comprises a borehole, further comprising measuring the temperature profile and measuring the measured signal characteristic at one of: (i) along a continuous length of the borehole; (ii) a single depth in the borehole; and (iii) a plurality of discrete depths in the borehole.

5. The method of claim 1, wherein the volume comprises a borehole, further comprising forming a loop in the optical fiber at a selected depth of the borehole and determining the partial pressure of hydrogen at the selected depth using the measured signal characteristic and a temperature at the selected depth obtained from the loop.

6. The method of claim 1, further comprising propagating a first light through the optical fiber at a first wavelength and a second light through the optical fiber at a second wavelength and determining a first hydrogen partial pressure based on the first light and a second hydrogen partial pressure based on the second light.

7. The method of claim 1, further comprising measuring the measured signal characteristic using Optical Time Domain Reflectometry (OTDR) and measuring the temperature profile using Distributed Temperature Sensing (DTS).

8. The method of claim 1, wherein the volume comprises a borehole, further comprising one of: (i) performing OTDR using a first optical interrogation unit and performing DTS using a second optical interrogation unit; (ii) performing OTDR using a single mode unit and performing DTS using a multimode unit; (iii) performing DTS along a first optical fiber in the borehole and performing OTDR along a second optical fiber in the borehole.

9. The method of claim 1, wherein the signal characteristic is a signal loss.

10. A system of determining a partial pressure of hydrogen in a volume, comprising:
an optical fiber;
a light source for generating a light at a selected wavelength for propagating through the optical fiber;
a light sensor for measuring the light after propagating through the optical fiber;
a processor configured to:
obtain a calibration signal characteristic along the optical fiber in an absence of hydrogen, wherein the calibration signal characteristic is associated with a presence of hydrogen;
measure a temperature profile along a section of the optical fiber with the optical fiber disposed in the volume;
obtain a measured signal characteristic along the section of the optical fiber with the optical fiber in the volume; and
determine the partial pressure of hydrogen in the volume from a comparison of the measured signal characteristic to the calibrated signal characteristic.

11. The system of claim 10, wherein the processor is further configured to determine the partial pressure of hydrogen from a ratio of a difference of the calibrated signal characteristic and the measured signal characteristic to a proportionality constant.

12. The system of claim 10, wherein the volume comprises a borehole and the processor is further configured to measure the temperature profile and obtain the measured signal characteristic at one of: (i) along a continuous length of the borehole; (ii) a single depth in the borehole; and (iii) a plurality of discrete depths in the borehole.

13. The system of claim 10, wherein the volume comprises a borehole and the optical fiber includes a loop at a selected depth of the borehole and wherein the processor is further configured to determine the partial pressure of hydrogen at the selected depth using the measured signal characteristic and the temperature at the selected depth obtained from the loop.

14. The system of claim 10, wherein the light source is further configured to propagate a first light through the optical fiber at a first wavelength and a second light through the optical fiber at a second wavelength and the processor is further configured to determine a first partial pressure of hydrogen based on the first light and a second partial pressure of hydrogen based on the second light.

15. The system of claim 10, wherein the processor is further configured to obtain the measured signal characteristic using Optical Time Domain Reflectometry (OTDR) and measure the temperature using Distributed Temperature Sensing (DTS).

16. The system of claim 10, wherein the volume comprises a borehole and the processor is further configured to perform one of: (i) OTDR using a first optical interrogation unit and DTS using a second optical interrogation unit; (ii) OTDR using a single mode unit and DTS using a multimode unit; (iii) DTS along a first optical fiber in the borehole and OTDR along a second optical fiber in the borehole.

17. A method of determining a presence of hydrogen in a volume, comprising:
measuring a response of a section of an optical fiber disposed in the volume to a parameter of the volume;
determining a partial pressure of hydrogen in the volume from the response of the optical fiber to the parameter; and
determining the presence of hydrogen in the volume from the partial pressure of hydrogen.

18. The method of claim 17, wherein determining the presence of hydrogen further comprises determining a concentration of hydrogen in the volume from the partial pressure of hydrogen.

19. The method of claim 18, further comprising measuring a temporal response of the optical fiber to the parameter and determining a temporal pattern of the hydrogen concentration in the volume from the temporal response.

20. The method of claim 17, wherein the parameter is one of: (i) temperature of the volume; and (ii) pressure of the volume.

21. A system for determining a partial pressure of hydrogen in a volume, comprising:
an optical fiber disposed in the volume;
a light source for generating a light at a selected wavelength for propagating through the optical fiber,
a light sensor for measuring the light after propagating through the optical fiber;
a processor configured to:
measure a response of a section of the optical fiber to a parameter of the volume;
determine the partial pressure of hydrogen in the volume from the response of the optical fiber to the parameter; and
determine a presence of hydrogen in the volume from the partial pressure of hydrogen.
